(19) United States
(12) Patent Application Publication
(10) Pub. No.: US 2023/0058936 A1
(43) Pub. Date: Feb. 23, 2023

(54) AUGMENTED REALITY SYSTEM FOR PHANTOM LIMB PAIN REHABILITATION FOR AMPUTEES

(71) Applicant: INTEGRUM AB, MÖLNDAL (SE)

(72) Inventor: Max Jair ORTIZ CATALAN, GÖTEBORG (SE)

(73) Assignee: INTEGRUM AB, MÖLNDAL (SE)

(21) Appl. No.: 17/977,585

(22) Filed: Oct. 31, 2022

Related U.S. Application Data
(63) Continuation of application No. 15/825,333, filed on Nov. 29, 2017, now abandoned.
Application No. 15/825,333 is a continuation of application No. 15/039,558, filed on May 26, 2016, now abandoned.

(30) Foreign Application Priority Data
Dec. 20, 2013 (SE) 1351570-5

(51) Int. Cl. G09B 5/02; A61F 2/72; A61B 5/11; A61B 5/00; A61B 5/389; G06F 3/01; G06T 11/00

(52) U.S. Cl. CPC G09B 5/02; A61F 2/72; A61B 5/1124; A61B 5/486; A61B 5/389; G06F 3/017; G06T 11/00; A61B 2505/09; A61F 2/60

(57) ABSTRACT

The present invention relates to a system for neuromuscular rehabilitation of a patient having an affected limb comprising: a feedback member arranged to give real-time visual feedback; a plurality of electrodes arranged to acquire an electric signal corresponding to an intent to move said affected limb; a control unit configured to: perform pattern recognition of said electric signals, wherein at least one feature in said electric signal is used to predict motion intent of said affected limb adjacent to at least one joint, such aggregated motions of said affected limb are predicted; based on output signals from said performed pattern recognition, control said feedback member to perform actions corresponding to said motions, whereby said actions of said feedback member are individually and simultaneously controlled by said patient via said intended motions.

### CROSS-REFERENCE TO RELATED APPLICATIONS

This application is a continuation of U.S. patent application Ser. No. 15/825,333, filed Nov. 29, 2017, which is a continuation of U.S. patent application Ser. No. 15/039,558, filed May 26, 2016 (now abandoned), which is a 371 U.S. National Stage of International Application No. PCT/SE2014/051546, filed Dec. 19, 2014, which claims priority to Swedish Patent Application No. 1351570-5, filed Dec. 20, 2013. The disclosures of each of the above applications are incorporated herein by reference in their entirety.

### FIELD OF THE INVENTION

The present invention relates to a system and a method for neuromuscular rehabilitation of a patient.

### BACKGROUND OF THE INVENTION

A successful rehabilitation is crucial for the quality of life after a traumatic event such as a limb amputation, which besides depriving a limb, in many cases results in phantom limb pain (PLP). Analogously, patients who have suffered stroke, incomplete spinal cord, and/or other nerve injuries, face neuromuscular rehabilitation challenges to alleviate what otherwise become life-permanent handicaps. PLP is a deteriorating condition commonly suffered by amputees. In fact, studies show that 70%-85% of amputees suffer from PLP. Traditionally, a method known as mirror therapy is used for relieving PLP. In mirror therapy, the motion of a healthy contralateral limb is mapped onto a representation of the missing limb such that the patient visually experiences motor execution in the missing limb by moving both limbs in the same manner.

The conventional mirror therapy method consists in placing a physical mirror in the plane separating the missing limb from the contralateral limb. This very crude type of mirror therapy can be replaced by more sophisticated methods and systems. For example, in the last decade, virtual reality (VR) has been employed as a more sophisticated version of conventional mirror therapy. Despite the advantages of employing VR over a traditional physical mirror, known VR approaches have retained the use of the contralateral limb and are therefore restricted to unilateral amputees. Promotion of motor execution (for example by producing phantom motions) in order to revert cortical reorganization, is the neuroscientific base of the mirror treatments. However, when using the contralateral limb as source of control, the actual effort made by the patient on the affected limb is disregarded, thus hindering the efficacy of the therapy.

One example of a VR based system is disclosed by U.S. Pat. No. 8,568,231 in which an entire virtual limb grossly moves based on motion sensors at the missing limb, rather than sensors on the contralateral limb. However, movements are determined from signals based on position data received from transmitters located at the location of e.g. a missing limb. Thereby, the number of possible motions is rather limited because an intended motion affects an entire virtual extension of the missing limb as a whole, rather than its individual components. A similar work is disclosed in “ReckAR, master's thesis by Morten Bak Kristoffersen, Aalborg University”.

Additional previous related work is described in “Evaluation of post-processing strategies for simultaneous pattern recognition based myoelectric prosthetic control (Joel Falk-Dahlin, master's thesis from Chalmers University, Sweden)”. However, as indicated by the title, this work was related to post-processing of prerecorded and available data.

In view of the above, there is a need for a system and method with improved promotion of motor execution for patients in need of neuromuscular rehabilitation, such as for example related to PLP.

### SUMMARY OF THE INVENTION

In view of the above-mentioned and other drawbacks of the prior art, a general object of the present invention is to provide a system and a method in which a virtual limb, computer games, and/or robotic devices, respond directly to the intention of motor execution decoded from bioelectric signals of the subject, and thus promoting motor control over the affected limb.

According to a first aspect of the invention there is provided a system for neuromuscular rehabilitation of a patient having an affected limb, the system comprising: a feedback member arranged to provide real-time visual feedback to the patient; a plurality of electrodes each arranged to acquire an electric signal generated from a portion of the patient's body, the at least one electric signal corresponding to an intent to move the affected limb; and a control unit configured to: perform pattern recognition of the electric signals, wherein at least one feature in the electric signal is used to predict motion intent of the affected limb adjacent to at least one joint, such that aggregated motions of the affected limb are predicted; and based on output signals from the performed pattern recognition, control the feedback member to perform actions corresponding to the aggregated motions, whereby the actions of the feedback member are individually and simultaneously controlled by the patient via the intended motions.

The present invention is based on the realization that promotion of motor execution enables an improved neuromuscular rehabilitation, and in the case of amputees, a reduction in PLP. Furthermore, the present invention is based on the realization that the motor execution is improved by enabling direct response of a virtual limb, games, and/or robotic devices, controlled via the predicted motions.

In the present context, an affected limb may be a disordered, missing, or an otherwise not fully functional limb. For example, an amputee has a missing limb which in the context of the present invention may be an affected limb. Furthermore, a patient who by any other means may not control a limb normally, for example as a result of incomplete spinal injury or stroke, may have an affected limb. In the present context, intent to move an affected limb refers to an attempt to move for example a malfunctioning limb or a missing limb.

In accordance with the invention, pattern recognition is the recognition of certain patterns in the electric signals acquired from a portion of the patient's body. For example by measuring an electric potential from a portion of a patient's body in the vicinity of the affected limb and predicting the intended motion according to the similarities of previously recorded patterns. Pattern of electric signals are characterized by signal features. The electric signals according to the invention may also be bioelectric signals.

In the present context an individual motion may be flexing of a joint, extending the joint, pronation, supination, open or close hand (in the case where a hand is part of an affected limb, and include several joints), ulnar deviation, radial deviation, or any other motion possible with a limb. The control unit thus extracts features indicative of an intended motion to feed a pattern recognition algorithm. Furthermore, an aggregated motion is the combination of one or more individual motions. For example, to reach for an object and grip the object with a hand, or grabbing an object with a hand and rotating it through supination of the hand, or open a hand while performing pronation of the hand. Furthermore, in the case of hand open/close, all the joints of the fingers are actuated together, thus resulting in aggregated motions (e.g. individual and simultaneous motions). In other words, several joints are performing individual motions at least partly simultaneously. Moreover, with aggregated motions the joints may be actuated individually, thus moving individual portions of the limb, or all together, for example with different speeds and ranges if necessary. Furthermore, an aggregated motion may be a first individual motion followed by a second individual motion.

In accordance with the invention, a feature may be for example signal variance, median frequency, root mean square, rate of pulse trains, or any other signal feature known in the art. A non-exhaustive list is of features in the time domain of an electrical signal is comprised in the following: mean absolute value, median, standard deviation, variance, waveform length, root mean square (RMS), zero-crossings of the signal, peaks (RMS), peaks mean, mean velocity, slope changes, power, difference absolute mean, max fractal length, fractal dimension, fractal dimension Higuchi, rough entropy, correlation, co-variance, etc. A non-exhaustive list is of features in the frequency domain of an electrical signal is comprised in the following: waveform length, mean, median, peaks mean, peaks median, peaks standard deviation, etc.

In accordance with the invention, neuromuscular rehabilitation comprises condition in which motor execution is required e.g. phantom limb pain, stroke, nerve and/or spinal cord injuries.

According to one embodiment of the invention, at least two aggregated motions of the affected limb may be predicted, wherein the control unit is configured to control the feedback member to perform at least two actions corresponding to the at least two motions.

According to one embodiment of the invention, the feedback member may be a robotic device. For example, the robotic device may be a remotely controlled vehicle, helicopter, prosthetic device or any other device which may be remotely controlled. A prosthetic device may be a prosthetic arm, leg, hand, foot, etc. Furthermore, the robotic device may comprise a sensor which measures a force applied to the sensor (thereby also to the robotic device). The sensor may send a signal to the control unit which may control an audio, visual, or tactile feedback about the presence or magnitude of the force. For example, the frequency or volume of an audio feedback may depend on the magnitude of the force, or the frequency or strength of the tactile feedback may depend on the magnitude of the force or may reflect the force measure by the force sensor. The force sensor may for example be a force sensor comprising e.g. a piezoelectric element, conductive polymer element or similar.

According to one embodiment of the invention, the system may further comprise a display arranged to provide the real-time visual feedback to the patient, the feedback member being a virtual limb, wherein the actions are motions performed by the virtual limb, such that the control unit is configured to: based on the output signals from the performed pattern recognition, control the virtual limb on the display to perform the motions, whereby the motions of the virtual limb are individually and simultaneously controlled by the patient via the intended motions. The virtual limb enhances the feeling for the patient of having a real arm, thus promoting motor execution further. In the present context, real-time may be delayed only by the control unit due to processing of the electric signals and controlling the virtual limb to perform the motions on the display.

According to one embodiment of the invention, the system may comprise a video capturing device arranged to acquire a video stream of the patient, the visual feedback comprises the video stream, wherein the control unit is configured to superimpose the virtual limb onto a predetermined portion of the patient's body in the visual feedback being displayed on the display, the virtual limb corresponding to the affected limb. By using an actual acquired image or video of the patient and superimposing a virtual limb in the image/video the illusion of a restored limb is enhanced. Thus, the patient will see him/herself on the display with a virtual arm in place of the affected or disordered limb. Superimposing a virtual limb into a real image enables an augmented reality. For example, when the patient intends to perform a motion in the affected limb, the virtual limb on the display moves according to the intended motion predicted via pattern recognition.

According to one embodiment of the invention, the virtual limb may follow the predetermined portion of the patient's body in the visual feedback being displayed on the display such that the virtual limb remains in an anatomically correct position. In other words, if the patient moves around, the virtual arm stays in the anatomically correct place where the affected (e.g. missing limb) should be. This way, the illusion of a restored limb is further enhanced. The virtual limb may follow the predetermined position by allowing the control unit to track, via the camera, fiducial markers arranged on the patient's affected limb in the position where the virtual limb is to be superimposed. A fiducial marker may for example be printed using a conventional printer, or the fiducial marker may be a light-emitting diode. Thereby, the virtual limb is superimposed on the marker as shown in the display, and the virtual limb changes scale and rotation based on the tracking of the marker.

According to one embodiment of the invention, for pattern recognition, the control unit may be configured to: divide each of the electric signals into signal segments defined by time intervals; extract signal features from the segments; combine the features into a feature vector relating to the motion; and based on the feature vector, predict the intended motion of the affected limb, wherein the features comprise an extracted cardinality of the data elements of the electrical signal. Accordingly, a feature vector may comprise at least one feature wherein one of the features is the cardinality feature. A cardinality of a vector is the number of unique elements of the vector. For example, if a signal is defined by the elements in the vector [1 1 2 3 5] the cardinality of the signals is four. Using the cardinality of the signal in each segment as a feature for pattern recognition, the accuracy of the pattern recognition is increased. As an example, the following is the average class-specific accuracy of a commonly used classifier (Linear Discriminant Analysis, LDA) in the prediction of 11 hand and wrist motions in 20 subjects when feed with the following features:

Cardinality: 90.0%

Zero crossings: 82.1%

Wave length: 82.0%

Root mean square value: 81.8%

Mean absolute value: 80.1%

Sign slope change: 74.7%

As can be seen from the above accuracies of prediction, the prediction accuracy when using the cardinality feature is higher compared to when using the other commonly used features listed above, resulting in an improved pattern recognition and thus prediction of intended motions.

For pattern recognition and thus prediction of an intended motion, a time window is first determined. For example, a time window may be 10-500 milliseconds, such as 200 milliseconds. The signals received by the control unit from the electrodes, one signal per electrode or electrodes pair, is divided or split in intervals of e.g. 200 milliseconds. One such interval is a segment. Thus, in accordance with the invention the electric signal is divided into segments where a segment is a portion of an electric signal defined by a certain time interval. The segments may or may not overlap. Moreover, at least one feature is extracted in a segment for each electrode (i.e. channel). For example, a first feature from the first channel and a second feature from the second channel etc. More than one feature per segment may be extracted and the type of feature extracted from different channels may be the same type of feature. The features extracted in the first segment of each channel are inserted into a vector known as a feature vector. In other words, a vector comprising all the features extracted during a time interval/segment. Algorithms used for pattern recognition may be e.g. supervised, unsupervised, and/or competitive, as well of a statistical nature (e.g. LDA), or biologically inspired (e.g. artificial neural networks, ANN). The algorithms may be arranged in different topologies (e.g. One-Vs.-One). The feature vector characterizes for example a motion.

In one embodiment of the present invention, the signal features are extracted from the time or frequency domain. For example, in the frequency domain one may more clearly identify the effect of fatigue, as fast fibers are not fatigue resistant.

According to one embodiment of the invention, the control unit is configured to, based on the output signals from the pattern recognition, control a video game on the display. A video game is an engaging tool for promoting motor execution since motivation increasing tasks may be implemented. For example, the feeling of completing a new level in a game or beating a competitor gives a patient a feeling of success, and thus a motivation for continuing the game while at the same time exercising motion in the affected limb. A video game may be specifically designed to promote motor execution of a particular degree of freedom. The video game is thus controlled by the patient through his/her intended motion of the affected limb. A video game may for example be a racing game, a fighting game, a platform game, etc.

According to one embodiment of the invention, the electrodes may be implantable into a patient's body for detecting bioelectrical signals. Using implantable electrodes is useful in case a patient is to control a prosthetic device, or for improved motor execution with direct contact with at least one nerve or muscle. Furthermore, an increased signal strength and signal-to-noise ratio may be possible which facilitates control over a virtual arm, games, or robotic device (e.g. prosthesis). Furthermore, with implantable electrodes a naturally perceived feedback by stimulating the nerves is possible. For example, with the implantable electrodes physiological feedback to the nerves may be provided such that the patient may for example experience the sensation of feeling an object using e.g. a prosthetic device.

Furthermore, the electrodes may be connected via an osseointegrated implant. For example, the implantable electrodes may be connected to the nerves via an osseointegrated implant.

In one embodiment, the visual feedback is in virtual reality. In other words, the entire visual feedback is virtually artificial with no real parts of the patient is included. Thus, a virtual world (not reflecting the real world) is shown on the display.

According to one embodiment of the invention, the electrodes are configured to measure myoelectric signals from the surface of the skin of the patient. This way, an easy manner to measure the (bio)electric signals is achieved by placing the electrodes on the surface of a patient's skin.

Furthermore, said control unit may be configured to, based on said output signals from said pattern recognition, control computer unit input commands. For example, the input commands may be to control the pointer on the computer unit, to press “return” or “space”, to open a web-browser, or any other software. Thus, the control unit may convert the signals acquired by the electrodes to input commands for the computer unit. The conversion may for example be made via a look-up table relating features of the acquired signals to input commands.

According to one embodiment of the invention, wherein said plurality of electrodes is a high density electrode array. In other words, the electrodes are placed close to each other such that signals are measured from the surface of the patient's skin from densely located points. This way, signals with sufficient quality may be found in a facilitated manner without the need for replacing the electrodes repeatedly. Furthermore, with such a high density array, a spatial filtering may be used to further improve the processing of the signals. For example, it may occur that two electrodes collect similar signals, then one of the signals may be disregarded, or the two signals may averaged to provide better single to noise ratio. Furthermore, blind signal separation may also be used for separation of signals within the set of signals provided by the plurality of electrodes. With a high density array, the individual electrode placement may be un-targeted, which means that they do not target a specific point on the surface of the limb. Instead, a control unit determines which electrodes collect useful data.

According to one embodiment of the invention, said plurality of electrodes may be arranged within a distance less than about 30 mm from each other, or less than about 20 mm from each, less than 10 mm or even less than 5 mm from each other.

According to one embodiment of the invention, said high density electrode array is arranged around an entire outer circumference of said affected limb. Thus, the electrode array may be arranged in a collar arrangement placed around the limb. With a large surface coverage of the high density array, an enhanced signal quality may be obtained.

According to one embodiment of the invention, wherein said control unit may further be configured to:

determine, by feature selection or signal separation, which of said plurality of electrodes are collecting useful data, and

discard or process data collected from electrodes determined not to collect useful data. Useful data may be data in which a known characteristic is recognized. Thus, if an electrode appears to collect data with e.g. high levels of noise, the signal from that electrode may be determined to be disregarded by the control unit. Furthermore, the control unit may recognize from the collected data whether an electrode is loose (e.g. “lead-off”) from the surface of the patient's skin. Feature selection relates to finding and selecting a set of features from the signals recorded by the electrode(s). “Brute force” algorithms or “Principal Component Analysis” can be used to select suitable sets of features which characterize better the bioelectric pattern of each movement found in the signal from an electrode.

According to one embodiment of the invention, further comprising a first and a second force applying element arranged to apply a respective first and second force to a respective first and second electrode in a direction towards a surface of said limb. The first and the second force may be maintained or changed in order to further improve the signal strength from the electrodes. The force applying means may be arranged on a stand with elongated first and second force applying elements directed towards respective electrodes. The patient may reach and adjust the force of the applied by the force applying elements in order to improve the signal from the respective electrode.

The electrodes may be implemented as a smart textile, which embeds dry electrodes with conductive leads paths to the control unit. This enables a simple and fast way of arranging the electrodes on the skin of the patient. For example, all the electrodes may be placed on the skin of the patient almost simultaneously by placing the smart textile with the electrodes in place on the patient on the limb.

Note that the embodiments mentioning specific electrode arrangements (e.g. high density array, force applying means, smart textile, and feature selection/signal separation) are applicable also independent from the other embodiments mentioned herein.

According to a second aspect of the invention, there is provided a method for controlling a system for neuromuscular rehabilitation of a patient having an affected limb, the method comprising the steps of: acquiring, via a plurality of electrodes, electric signals generated from a portion of the patient's body, the at least one electric signal corresponding to an intent to move the affected limb; performing pattern recognition of the electric signals; predicting motion intent in at least one joint using at least one feature in the electric signal, such that aggregated motions of the affected limb are predicted; and based on output signals from the performed pattern recognition, controlling a feedback member to perform actions corresponding to the motions, whereby the actions of the feedback member are individually and simultaneously controlled by the patient via the intended motions.

According to one embodiment of the invention, the feedback member may be a virtual limb shown on a display for providing the visual feedback, wherein the actions are motions performed by the virtual limb, such that the method comprises: based on the output signals from the performed pattern recognition, controlling the virtual limb on the display to perform the motions, whereby the motions of the virtual limb are individually and simultaneously controlled by the patient via the intended motions.

According to one embodiment of the invention, the method may comprise the step of superimposing the virtual limb onto a predetermined portion of the patient's body in the visual feedback being displayed on the display, the virtual limb corresponding to the affected limb.

According to one embodiment of the invention, the pattern recognition comprises the steps of: dividing each of the electric signals into signal segments defined by time windows; extracting signal features from at least one of the segments; combining the features into a feature vector; and based on the feature vector, predicting the intended motion of the affected limb, wherein the features comprise the cardinality of data elements of the electrical signal. Moreover, the cardinality may be used, exclusively, or in combination with other time and frequency domain features.

According to one embodiment of the invention, the method may comprise the steps of: executing predetermined motions predefined by the control unit; associating the features in the electric signals with the executed predetermined motions; performing rehabilitation tasks by the patient, wherein the tasks are training motions predetermined in the control unit; and reporting the patient's progress on the display. The training motion may for example be individual or simultaneous joint actuations (e.g. hand open/close). Reporting may be done in an electronic questionnaire presented on the display. Furthermore, reporting may be performed via predicted intended motions by the patient and may comprise a description by the patient on the experience of the performing the tasks, as well as the performance in the tasks. Reporting may for example comprise entering the amount of pain experienced by the patient during a predetermined time. For example, the progression of the pain may be reported. Furthermore, the prior to reporting the patient's progress, the control may predict the training motions based the associated features, wherein said feedback member performs said training motions.

Effects and features of this second aspect of the present invention are largely analogous to those described above in connection with the first aspect of the invention.

According to a third aspect of the invention there is provided a computer program product comprising a computer readable medium having stored thereon computer program means for a system for neuromuscular rehabilitation of a patient having an affected limb, the system comprising a control unit, wherein the computer program product comprises: code for acquiring, via a plurality of electrodes, electric signals generated from a portion of the patient's body, the at least one electric signal corresponding to an intent to move the affected limb; code for performing pattern recognition of the electric signals; code for predicting motion intent in at least one joint using at least one feature in the electric signal, such that aggregated motions of the affected limb are predicted; and code for, based on output signals from the performed pattern recognition, controlling a feedback member to perform actions corresponding to the motions, whereby the actions of the feedback member are individually and simultaneously controlled by the patient via the intended motions.

Effects and features of this third aspect of the present invention are largely analogous to those described above in connection with the first and second aspects of the invention.

According to a fourth aspect of the present invention, there is provided a method for controlling an artificial limb comprising the steps of: acquiring an electrical signal generated from a portion of a patient's body; during acquisition, dividing the electric signals into signal segments defined by time windows; extracting signal features from at least one of the segments; forming a feature vector comprising the features; wherein the extracting features comprise at least the cardinality of a vector comprising data elements corresponding to the electrical signal in at least one segment.

A cardinality of a vector is the number of unique elements. For example, if a signal is defined by the elements in the vector [1 1 2 3 6] the cardinality of the signals is four. Using the cardinality of the signal in each segment as a feature for pattern recognition the accuracy of the pattern recognition is increased. The cardinality could for example be computed by sorting the data elements of a segment and delete repeated elements, then count the remaining data elements. The feature of cardinality has been realized to enhance the capability to predict individual and simultaneous motions.

According to one embodiment of the invention, the artificial limb may be a prosthetic device. For example, a prosthetic device may be a prosthetic arm, leg, hand, foot, etc.

According to one embodiment of the invention, the artificial limb may be a robotic device. For example, a remotely controlled vehicle, helicopter, or any other device which may be remotely controlled.

According to one embodiment of the invention, the robotic device may be controlled by a patient via intended motions actuated by a joint adjacent to a limb of the patient to perform at least two actions.

According to one embodiment of the invention, the artificial limb may be a virtual limb displayed on a display. For example, the virtual limb may be part of a virtual reality or augmented reality shown to the patient on the display as a real-time visual feedback.

According to one embodiment of the invention, the electrical signals are acquired via surface or implantable electrodes. For example, implantable electrodes may be implantable into a patient's body and be arranged to connect with the nerves and/or muscles of the patient.

Effects and features of this fourth aspect of the present invention are largely analogous to those described above in connection with the previous aspects of the invention.

According to a fifth aspect of the invention, there is provided for use of a system for neuromuscular rehabilitation of a patient having an affected limb, said system comprising:

a feedback member arranged to provide real-time visual feedback to said patient;

a plurality of electrodes each arranged to acquire an electric signal generated from a portion of said patient's body, said at least one electric signal corresponding to an intent to move said affected limb; and a control unit configured to:

perform pattern recognition of said electric signals, wherein at least one feature in said electric signal is used to predict motion intent of said affected limb adjacent to at least one joint, such that aggregated motions of said affected limb are predicted; and

based on output signals from said performed pattern recognition, control said feedback member to perform actions corresponding to said aggregated motions, whereby said actions of said feedback member are individually and simultaneously controlled by said patient via said intended motions, wherein said use comprises:

executing predetermined motions predefined by said control unit; associating said features in said electric signals with said executed predetermined motions;

performing rehabilitation tasks by said patient, wherein said tasks are training motions predetermined in said control unit;

predicting said training motions based on the associated features and on the acquired signals from said portion of the patients body;

wherein said feedback member performs said predicted training motions; and

reporting said patient's progress on said display.

Thus, first the patient executes motion such that the control unit may associate features in the signals with the motions. Next, the patient performs training motions. The control unit collects electric signals from the electrodes and based on the signals and on the associated features, the control unit predicts the training motions. The feedback member performs the predicted training motions. Reporting may be to report the progression of pain felt by the patient in the limb (e.g. phantom pain) or the overall repeatability of the predicted training motions (i.e. if the predicted training motions correspond with the performed motions). The reporting may be stored in the control unit for further processing of follow up b e.g. a medical expert.

Effects and features of this fifth aspect of the present invention are largely analogous to those described above in connection with the previous aspects of the invention.

Further features of, and advantages with, the present invention will become apparent when studying the appended claims and the following description. The skilled person realize that different features of the present invention may be combined to create embodiments other than those described in the following, without departing from the scope of the present invention.

### BRIEF DESCRIPTION OF THE DRAWINGS

FIG. 1 illustrates a schematic drawing of a system according to an alternative embodiment of the invention;

FIG. 2 illustrates a schematic drawing of a system according to an alternative embodiment of the invention;

FIG. 3 illustrates exemplary individual motions of a limb;

FIG. 4 illustrates a flow-chart of an alternative embodiment according to the invention;

FIG. 5 illustrates a flow-chart of an alternative embodiment according to the invention; and

FIG. 6 illustrates an exemplary electrode placement.

### DESCRIPTION OF EXAMPLE EMBODIMENTS OF THE PRESENT INVENTION

In the following description, the present invention is mainly described with reference to neuromuscular rehabilitation in relation to phantom limb pain in a patient with a missing limb. It should, however, be noted that this by no means limits the scope of the invention, which is equally applicable to neuromuscular rehabilitation of any other condition where myoelectric and/or neuroelectric signals can be used to drive the system and promote motor execution. For example, such as neuromuscular rehabilitation of a patient suffering from e.g. stroke, incomplete spinal cord, and/or nerve injuries.

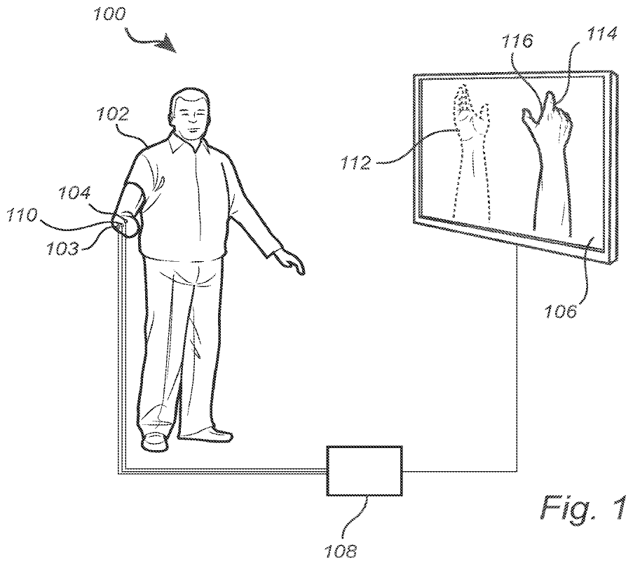

FIG. 1 illustrates a schematic of a system 100 according to an exemplary embodiment of the invention. FIG. 1 shows a patient 102 having an affected limb 104, the patient 102 is using the system 100. In this case the affected limb 104 has a missing portion such that there is missing limb, for example as a result of an amputation. Furthermore, FIG. 1 shows a display 106 connected to a control unit 108 and a plurality of electrodes 110 also connected to the control unit 108. Although three electrodes are shown in FIG. 1, any suitable number of electrodes is possible to use. The connection between the display 106 and the control unit 108 may be enabled through physical wires or by wireless connection. The electrodes 110 are arranged to acquire an electric signal generated from a portion 103 of the patient's body, in this particular case the stump 103 of a limb having a missing portion. As illustrated in this exemplary embodiment, the electrodes 110 are attached on the surface of the patient's skin such that myoelectric signals may be recorded from the patient. However, the electrodes 110 may also be implantable electrodes 110. The control unit 108 is configured to read electric signals acquired by the electrodes 110 and perform pattern recognition of the electric signals. The control unit 108 processes the electric signals and recognizes features in the electric signals such that motion intent of aggregated motions is predicted. In other words, the control unit 108 predicts motions of the missing limb intended by the patient 102. After the intended motion has been predicted, the control unit 108 controls the virtual limb 116 on the display to perform the intended motions. For example, if a patient 102 intends to move his limb at a first joint, for example the elbow, and at the same time supinate the hand such that it rotates, the virtual limb 116 will move from a first position 112 to a second position 114 according to the aggregated intended motions. In the first position the missing limb is to the left and the palm of the hand faces the patient 102 viewing the display, then the aggregated motion of the limb intended by the patient 102 is to flex the elbow joint and to pronate the hand, the intended motion is predicted by the control unit 108 and the limb moves to position 114. Other motions are of course possible such as for example flexing of a limb, extending the limb, pronation, supination, open or close hand (in the case where a hand is part of an affected limb), or any other motion possible with a limb, and/or combinations thereof. The control unit 108 may be connected to the display 106 by a wireless connection or via physical electrical connections. Moreover, although in FIG. 1 a virtual arm is shown, a robotic device may be used instead. For example, the display with the virtual arm may be replaced by a prosthetic device or a remotely controlled vehicle or similar.

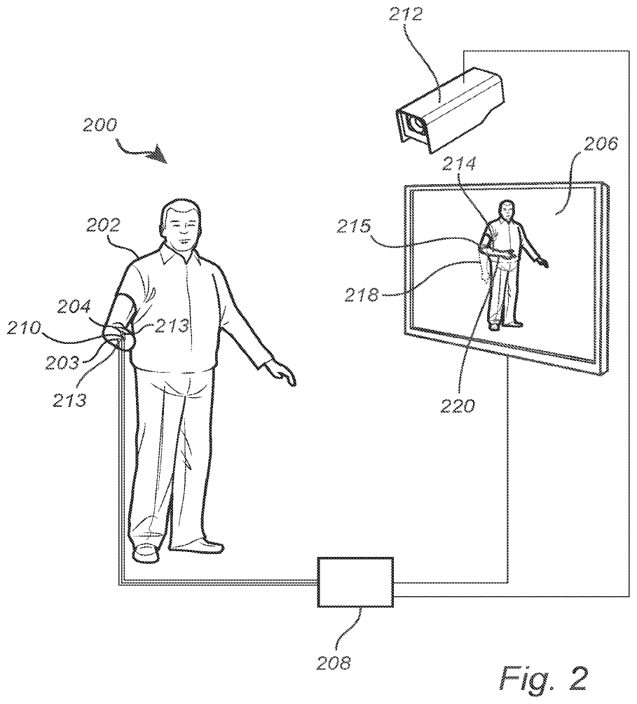

FIG. 2 shows another system 200 according to an embodiment of the invention. Similar to what is depicted in FIG. 1, FIG. 2 shows a patient 202 having an amputated limb 204, and the patient 202 is using the system 200. At the stump 203 of the amputated limb there is a plurality of electrodes 210 arranged to acquire an electric signal at the stump 203. Furthermore, the electrodes 210 are connected to a control unit 208 and there is also a display 206 connected to the control unit 208. Although three electrodes are shown in FIG. 2, any suitable number of electrodes is possible. The display 206 is configured to provide real-time feedback to the patient 202 and to show a virtual limb 214. Furthermore, there is a video capturing device in the form of a camera 212 arranged to acquire a video stream of the patient 202. The camera 212 may be a conventional webcam. For example the camera 212 may be a simple traditional webcam. Moreover, the video stream is shown on the display 206. In other words the patient 202 can see him/herself in real-time on the display 206. The control unit 208 is configured to superimpose the virtual limb 214 onto a predetermined portion 215 of the patient's body shown on the display 206. As shown in FIG. 2, the virtual limb 214 is a portion of an arm and the hand of the arm, superimposed onto the stump 215 of the affected limb being an amputated limb. Furthermore, there are markers 213 such as fiducial markers 213 arranged on the stump of the amputated limb on the patient such that the control unit 208 may track the motion of the affected limb 204 via the webcam 212. This enables the virtual limb 214 to be aligned anatomically correctly with the stump 215 of the amputated limb of the patient 216 on the display. Moreover, if the patient 202 moves around, the virtual limb 214 stays in the anatomically correct position on the display 206. Similar to what is described with reference to FIG. 1, the control unit 208 is arranged to read electric signals acquired by the electrodes 210 and to perform pattern recognition on the electric signals. The control unit 208 processes the electric signals and recognizes features in the electric signals such that motion intent of aggregated motions is predicted. In other words, the control unit 208 predicts aggregated motions of the missing limb intended by the patient. After the intended motions have been predicted, the control unit 208 controls the virtual limb 214 on the display to perform the intended motions. For example, if a patient intends to move his limb at a first joint, for example the elbow, and at the same time supinate the hand such that it rotates, the virtual limb will move from a first position 218 to a second position 220 according to the intended motion. In the first position 218 the missing limb is to the left and the palm of the hand faces away from the patient 202 viewing the display 206, then the motion of the limb intended by the patient is to flex the elbow joint and to supinate the hand, the intended motion is predicted by the control unit 208 and the virtual limb moves to position 220. Other motions are of course possible, such as for example flexing of a limb, extending the limb, pronation, supination, open or close hand (in the case where a hand is part of an affected limb), or any other motion possible with a limb, and/or combinations thereof. The control unit 208 may be connected to the display 206 by a wireless connection or via physical electrical connections.

Superimposing of the virtual limb onto a predetermined portion of the patient's body as described with reference to FIG. 2 enables an augmented reality. Furthermore, the patient may play a video game shown on the display via intended motions. In other words, the conventional mouse and keyboard are substituted by predicted motions.

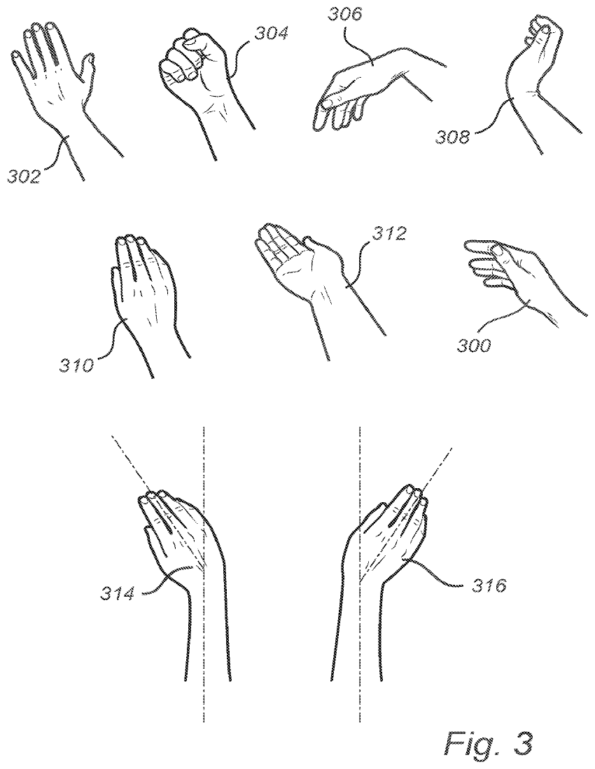

FIG. 3 illustrates exemplary individual motions of a limb. In this case the limb is a hand, however, the limb may within the context of the invention be any limb such as an arm, a leg, a foot, etc. In FIG. 3 a limb, illustrated as a hand, is shown in a resting position 300, and the other positions 302-316 are relative to the resting position 300. The other positions are: open hand 302, closed hand 304, flexed position 306, extended position 308, pronation 310, supination 312, ulnar deviation 314, and radial deviation 316. Note that other motions are also possible. For example, it is possible to move the hand in any of the above motions at the same time as performing any motion with the arm adjacent to the elbow.

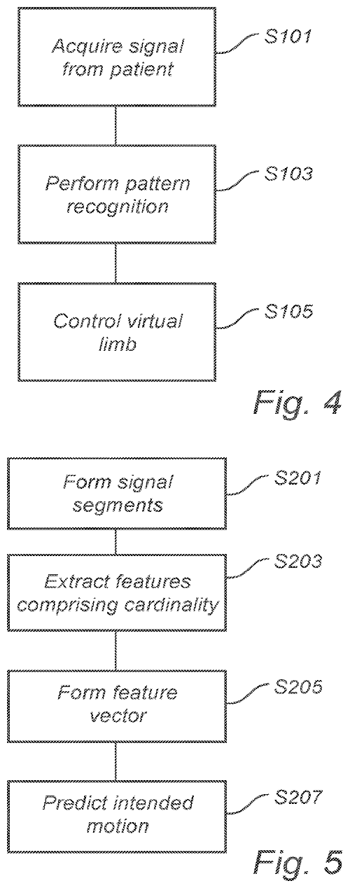

FIG. 4 is a flow-chart illustrating the method steps according to an embodiment of the invention. The method may be implemented using for example the system depicted in FIG. 1 or FIG. 2. In a first step S101 electrical signals are acquired from a patient. The electrical signals may for example be acquired from surface electrodes mounted on the surface of the skin of the patient or may be acquired from electrodes implanted into the patient and connected to nerves of the patient. In a second step S103, pattern recognition is performed on the electrical signals acquired by the electrodes. During pattern recognition, features of the electric signals are distinguished and determined to correspond to certain motions of the limb intended by the patient. In a final step S105, a virtual limb is controlled to perform motions based on the predicted motions. The virtual limb is displayed on a display shown to the patient. Optionally, a robotic device is controlled via the pattern recognition, for example a prosthetic device may be controlled.

FIG. 5 is a flow-chart illustrating the method steps according to another embodiment of the invention. In FIG. 5 the pattern recognition S103 steps are illustrated in more detail. In a first step S201, the electrical signals acquired from the patient are divided into segments. The length of the segments is defined in terms of predetermined time windows. For example, a time window may have 200 ms duration. Moreover, each electrical signal from the independent electrodes is divided into segments. Within each segment at least one characteristic features is extracted. A non-exhaustive list is of features of in the time domain of an electrical signal is comprised in the following: mean absolute value, median, standard deviation, variance, waveform length, root mean square (RMS), zero-crossings of the signal, peaks (RMS), peaks mean, mean velocity, slope changes, power, difference absolute mean, max fractal length, fractal dimension, fractal dimension Higuchi, rough entropy, correlation, co-variance, etc. A non-exhaustive list is of features in the frequency domain of an electrical signal is comprised in the following: waveform length, mean, median, peaks mean, peaks median, peaks standard deviation, etc. Furthermore, the cardinality of the segments is extracted as a characteristic feature. The cardinality of a signal is the number of unique elements of the signal. For example, if a signal is defined by the elements in the vector [1 1 2 3 5] the cardinality of the signals is four. Using the cardinality of the signal in each segment as a feature for pattern recognition, the accuracy of the pattern recognition is increased. As an example, the following is the average class-specific accuracy of a commonly used classifier (Linear Discriminant Analysis, LDA) in the prediction of 11 hand and wrist motions in 20 subjects when fed with the following features:

Cardinality: 90.0%

Zero crossings: 82.1%

Wave length: 82.0%

Root mean square value: 81.8%

Mean absolute value: 80.1%

Sign slope change: 74.7%

As can be seen from the above accuracies of prediction, the prediction accuracy when using the cardinality feature is higher compared to when using the other commonly used features listed above, resulting in an improved pattern recognition and thus prediction of intended motions.

In a subsequent step S205, a feature vector is formed by placing the extracted features, including the cardinality, as entries in a vector for the present segment. From the feature vector, an intended motion is predicted S207 using known algorithms. Algorithms used for pattern recognition may be e.g. supervised, unsupervised, and/or competitive, as well of a statistical nature (e.g. LDA), or biologically inspired (e.g. artificial neural networks, ANN). The algorithms may be arranged in different topologies (e.g. One-Vs.-One).

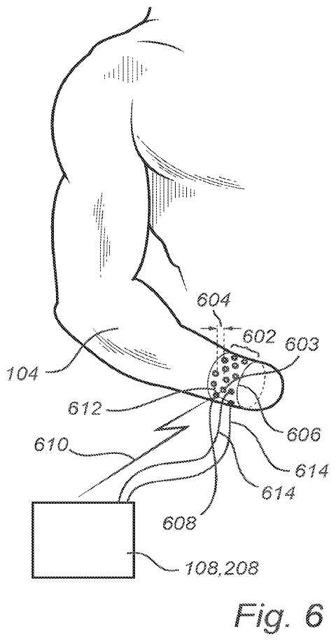

FIG. 6 illustrates an array of electrodes arranged on a limb of a person, for example the limb may be an affected limb 104. On the skin of the limb there is a high density array 602 of electrodes 603 (only one is numbered). The number of electrodes is at least six, but may be for example, 8, 10, 12 or more. The electrodes 603 are arranged close to each other in order to densely cover a large area of the limb. For example, the distance 604 between adjacent electrodes may be less than 30 mm, or even less such as less than 10 mm or less than 5 mm. With such a high density array 602, the placement of the individual electrodes 603 is less crucial, instead a control unit 108, 208, 608 may determine which signals are useful. The control unit 608 may be arranged adjacent to the electrodes 603 and may communicate wirelessly 610 with the control unit 108, 208. Alternatively, or additionally the control unit 108, 208 communicates directly with the electrodes via cables 614. In one example embodiment, the array 602 of electrodes extends around the outer circumference 606 of the limb in order to cover a large area around the limb 104. The control unit 108, 208, or 608 may determine from the signals acquired from the electrodes 603 of the array 602 which of the signals are useful. For example, the control unit 108, 208, or 608 may use a feature selection algorithm or a signal separation algorithm to determine if a signal is useful. The electrode arrangement embodiment shown in FIG. 6 may be used separate from the other embodiments described herein.

Additionally, variations to the disclosed embodiments can be understood and effected by the skilled person in practicing the claimed invention, from a study of the drawings, the disclosure, and the appended claims. For example, virtual reality is also possible within the context of the invention although only augmented reality is shown in the exemplary embodiment of FIG. 2. The system and method may further be used for controlling a robotic device such as a remotely controlled toy vehicle, helicopter, boat, robotic arm such as for example a prosthesis, etc. Furthermore, although three electrodes are shown in FIG. 1 and FIG. 2, any suitable number of electrodes is possible within the scope of the invention.

Furthermore, the methods disclosed and described may be used by a person not having an affected limb, thus the method and the system is equally applicable to a person having only non-affected limbs.

Furthermore, the communication between electrodes and the control unit may be either via wires of via wireless communication. In case of wireless communication, there may be a second control unit arranged nearby the electrodes.

In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

What is claimed is:

1. A phantom limb pain rehabilitation augmented reality system for rehabilitation of a patient having an amputated limb, said system comprising:
a video capturing device arranged to face towards said patient and to acquire a video stream capturing at least a majority of said patient including a stump of the amputated limb;
a display arranged to provide a real-time visual feedback to said patient, the real-time visual feedback comprising the video stream and a virtual limb in place of a missing portion of the amputated limb, wherein the at least the majority of the patient, the stump, and the virtual limb are displayed in the real-time visual feedback;
a plurality of electrodes each arranged to acquire an electric signal generated from the stump of the amputated limb, or from a portion of the patient's body on the same side as the amputated limb, said electric signal corresponding to an intent to move said missing portion being associated with phantom limb pain; and
a control unit configured to:
perform pattern recognition of said electric signals, wherein at least one feature in said electric signals is used to predict motion intent of said missing portion of the amputated limb adjacent to at least one joint, to thereby predict aggregated motions comprising individual motions of several joints of said missing portion of the amputated limb performed at least partly simultaneously to alleviate the phantom limb pain;
track the motion of the stump in the visual feedback being displayed on said display;
superimpose said virtual limb onto the stump of said patient's body in said visual feedback being displayed on said display, wherein said virtual limb follows said stump of said patient's body in said visual feedback being displayed on said display such that said virtual limb remains in an anatomically correct position;
based on output signals from said performed pattern recognition, control said virtual limb on said display to perform motions corresponding to said aggregated motions to provide the phantom limb pain rehabilitation for the patient, whereby said motions of said virtual limb are individually and simultaneously controlled by said patient via said intended motions,
wherein, for pattern recognition, said control unit is configured to:
divide each of said electric signals into signal segments defined by time intervals;
extract signal features from at least one of said segments;
combine said signal features into a feature vector relating to said motion; and
based on said feature vector, predict said intended motion of said missing portion of the amputated limb.

2. The system according to claim 1, wherein at least two aggregated motions of said missing portion of the amputated limb are predicted, wherein said control unit is configured to control said virtual limb to perform at least two actions corresponding to said at least two motions.

3. The system according to claim 1, wherein the control unit is configured to extract signal features from each of said segments, and wherein said features comprise, for each of said segments, an extracted cardinality of data elements within a segment of said electrical signal.

4. The system according to claim 1, wherein said control unit is configured to, based on said output signals from said pattern recognition, control a video game on said display.

5. The system according to claim 1, wherein said electrodes are implantable into a patient's body for detecting bioelectrical signals.

6. The system according to claim 1, wherein said control unit is configured to, based on said output signals from said pattern recognition, control computer unit input commands.

7. The system according to claim 1, wherein said plurality of electrodes is a high density electrode array.

8. The system according to claim 1, wherein the individual motions are at least two of flexing of a joint, extending the joint, pronation, supination, open or close hand, ulnar deviation, and radial deviation.

9. The system according to claim 7, wherein said control unit is further configured to:
determine, by feature selection or signal separation, which of said plurality of electrodes are collecting useful data, and
discard or process data collected from electrodes determined not to collect useful data.

10. The system according to claim 1, wherein the display is a desktop display screen.

11. The system according to claim 1, wherein tracking of the predetermined portion of the patient's body is performed by tracking markers arranged on the predetermined portion of the patient's body.

12. The system according to claim 1, wherein the control unit receives and stores a report of the progression of phantom pain felt by the patient.

13. A method for controlling a phantom limb pain rehabilitation augmented reality system for rehabilitation of a patient having an amputated limb, said method comprising the steps of:
acquiring, via a plurality of electrodes, electric signals generated from a stump of the amputated limb or from a portion of the patient's body on the same side as the amputated limb, said electric signals corresponding to an intent to move a missing portion of the amputated limb being associated with phantom limb pain;
performing pattern recognition of said electric signals;
predicting motion intent in at least one joint using at least one feature in said electric signals, to thereby predict aggregated motions comprising individual motions of several joints of said missing portion of the amputated limb performed at least partly simultaneously to alleviate the phantom limb pain;
providing real-time visual feedback to said patient on a display, the real-time visual feedback comprising a video stream of said patient, captured by a video capturing device facing towards the patient, and a virtual limb corresponding to said missing portion of the amputated limb such that at least a majority of the patient, the stump, and the virtual limb are displayed in the real-time visual feedback;
tracking the motion of a stump of the patient's body in the visual feedback being displayed to the patient;
superimposing said virtual limb onto the stump of said patient's body in said visual feedback, wherein said virtual limb follows said stump of said patient's body in said visual feedback such that said virtual limb remains in an anatomically correct position on the stump; and
based on output signals from said performed pattern recognition, controlling the virtual limb to perform motions corresponding to said aggregated motions to provide the phantom limb pain rehabilitation for the patient, whereby said motions of said virtual limb are individually and simultaneously controlled by said patient via said intended motions,
wherein said pattern recognition comprises:
dividing each of said electric signals into signal segments defined by time windows,
extracting signal features from at least one of said segments,
combining said signal features into a feature vector, and
based on the feature vector, predicting said intended motion of said stump and missing portion of the amputated limb.

14. The method according to claim 13, wherein said pattern recognition comprises extracting signal features from each of said signal segments, and wherein said features comprise, for each of said segments, an extracted cardinality of data elements within a segment of said electrical signal.

15. The method according to claim 13, comprising the steps of:
executing predetermined motions predefined by said control unit;
associating said features in said electric signals with said executed predetermined motions;
performing rehabilitation tasks by said patient, wherein said tasks are training motions predetermined in said control unit; and
reporting said patient's progress on said display.

16. The method according to claim 13, wherein reporting the patient's progress includes reporting the amount of pain experienced by the patient.

17. Use of a phantom limb pain rehabilitation system for rehabilitation of a patient having an amputated limb, said system comprising:
a video capturing device arranged to face towards said patient and to acquire a video stream capturing at least a majority of said patient including a stump of the amputated limb;
a display arranged to provide a real-time visual feedback to said patient, the real-time visual feedback comprising the video stream and a virtual limb in place of a missing portion of the amputated limb, wherein the at least the majority of the patient, the stump, and the virtual limb are displayed in the real-time visual feedback;
a plurality of electrodes each arranged to acquire an electric signal generated from the stump of the amputated limb, or from a portion of the patient's body on the same side as the amputated limb, said electric signal corresponding to an intent to move said missing portion being associated with phantom limb pain; and
a control unit configured to:
perform pattern recognition of said electric signal, wherein at least one feature in said electric signal is used to predict motion intent in the missing portion of the amputated limb adjacent to at least one joint, to thereby predict aggregated motions comprising individual motions of several joints of said missing portion of the amputated limb performed at least partly simultaneously to alleviate the phantom limb pain;
track the motion of a stump of the patient's body in the visual feedback being displayed on said display;
superimpose said virtual limb onto the stump of said patient's body in said visual feedback being displayed on said display, wherein said virtual limb follows said stump of said patient's body in said visual feedback being displayed on said display such that said virtual limb remains in an anatomically correct position;
based on output signals from said performed pattern recognition, control said virtual limb to perform motions corresponding to said aggregated motions to provide the phantom limb pain rehabilitation for the patient, whereby said actions of said virtual limb are individually and simultaneously controlled by said patient via said intended motions,
wherein, for pattern recognition, said control unit is configured to:
divide each of said electric signals into signal segments defined by time intervals;
extract signal features from at least one of said segments;
combine said signal features into a feature vector relating to said motion; and
based on said feature vector, predict said intended motion of said missing portion of the amputated limb, and
wherein said use comprises:
executing predetermined motions predefined by said control unit;
associating said features in said electric signals with said executed predetermined motions;
performing rehabilitation tasks by said patient, wherein said tasks are training motions predetermined in said control unit;
predicting said training motions based on the associated features and on the acquired electric signals from said portion of the patient's body, wherein said virtual limb performs said predicted training motions; and
reporting said patient's progress on said display.

18. Use according to claim 17, wherein reporting includes to report the progression of phantom pain felt by the patient, the reporting is stored in the control unit.

19. The system according to claim 1, wherein said features further comprise at least one of:
with respect to time domain:
mean absolute value, median, standard deviation, variance, waveform length, root mean square (RMS), zero-crossings of the signal, peaks (RMS), peaks mean, mean velocity, slope changes, power, difference absolute mean, max fractal length, fractal dimension, fractal dimension Higuchi, rough entropy, correlation, and co-variance; and
with respect to frequency domain:
waveform length, mean, median frequency, peaks mean, peaks median, and peaks standard deviation.

20. The method according to claim 13, wherein said features further comprise at least one of:
with respect to time domain:
mean absolute value, median, standard deviation, variance, waveform length, root mean square (RMS), zero-crossings of the signal, peaks (RMS), peaks mean, mean velocity, slope changes, power, difference absolute mean, max fractal length, fractal dimension, fractal dimension Higuchi, rough entropy, correlation, and co-variance; and
with respect to frequency domain:
waveform length, mean, median frequency, peaks mean, peaks median, and peaks standard deviation.
